(19) United States
(12) Patent Application Publication
(10) Pub. No.: US 2025/0161223 A1
(43) Pub. Date: May 22, 2025

(54) PHARMACEUTICAL FORMULATION

(71) Applicant: Novartis AG, Basel (CH)

(72) Inventors: Bindhumadhavan GURURAJAN, Basel (CH); Rohit LOWALEKAR, Hyderabad (IN); Yogesh PAWAR, Hyderabad (IN); Emeric REYNAUD, Village Neuf (FR); Vincent ROGUE, Bouxwiller (FR); Tushar SAWAI, Hyderabad (IN)

(21) Appl. No.: 18/839,600

(22) PCT Filed: Feb. 20, 2023
(86) PCT No.: PCT/IB2023/051532

§ 371 Date: Aug. 19, 2024

(30) Foreign Application Priority Data
Feb. 21, 2022 (IN) 202211009181

(51) Int. Cl. A61K 9/28; A61K 9/14; A61K 9/20; A61K 31/506; A61K 45/06

(52) U.S. Cl. CPC A61K 9/2866; A61K 9/145; A61K 9/146; A61K 9/2009; A61K 9/2018; A61K 9/2054; A61K 9/2826; A61K 9/284; A61K 9/2853; A61K 31/506; A61K 45/06

(57) ABSTRACT

The invention relates to a pharmaceutical formulation comprising the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and at least one pharmaceutically acceptable excipient, where, in particular, the pharmaceutical formulation is made by a process comprising wet granulation, direct compression or especially roller compaction, and related invention aspects disclosed herein.

### BACKGROUND

### Field of the Invention

The invention relates to a formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and different aspects and further invention embodiments associated with this formulation and its manufacture, as provided in more detail below and in the claims.

### Background of the Invention

(3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155) is an orally bioavailable, allosteric inhibitor of Src homology-2 domain containing protein tyrosine phosphatase-2 (SHP2, encoded by the PTPN11 gene), which transduces signals from activated receptor tyrosine kinases (RTKs) to downstream pathways, including the mitogen-activated protein kinase (MAPK) pathway, the JAK-STAT and the phosphoinositol 3-kinase (PI3K)-AKT pathways. SHP2 has also been implicated in immune checkpoint and cytokine receptor signaling. TNO155 has demonstrated efficacy in a wide range of RTK-dependent human cancer cell lines and in vivo tumor xenografts. Details of TNO155, the pharmacological activity and other properties thereof and its manufacture, as well as salts and polymorphs thereof, can be found in, for example, WO 2015/107495A, WO 2020/165734A, WO 2020/065452A and WO 2020/065453 which relates, in part, to the succinate salt of TNO155, as Modification (form) H_{A} of the succinate (1:1) hemihydrate of TNO155 (Example 6) and other polymorphs. For TNO155, the hydrate form Modification H_{A} of the succinate salt is more stable than the anhydrous form, and form H_{A} is the active ingredient comprised in the present formulations.

SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors leads to exposure of the catalytic site resulting in enzymatic activation of SHP2.

Mutations in the PTPN11 gene and subsequently in SHP2 have been identified in several human diseases, such as, but not limited to, Noonan Syndrome, Leopard Syndrome, juvenile myelomonocytic leukemias, neuroblastoma, melanoma, acute myeloid leukemia and cancers of the breast, lung and colon. SHP2, therefore, represents a highly attractive target for the development of novel therapies for the treatment of various (especially proliferative) diseases. The pharmaceutical formulations that can be manufactured according to the present invention fulfil the need to inhibit the activity of SHP2.

For the manufacturing of tablets, there are basically three methods that exist in principle: wet granulation and dry granulation in the form of slugging or especially roller compaction for the formulation of granules that are then compacted, or directly compressed. The resulting granulates can then be pressed into tablets. Dry granulated granulates and tablets formed with them are fundamentally different in their structure to wet granulated granules and tablets.

Wet granulation (WG) involves converting a powder mixture into granules before mixing with other excipients and compressing the mixture to form a tablet. The drug is typically mixed with a granulating fluid (for example, aqueous or alcoholic) and a binder to form a granulation mixture. Other excipients may also be included in the granulation mixture. The binder helps to bond powder particles of the drug together, since many drugs have poor cohesive properties. The granulation mixture is then dried to remove the solvent, resulting in granules in which drug particles are bound together with a binder and any other excipients present in the granulation mixture. These granules are mixed with other excipients and compressed into a tablet.

Direct compression (DC) allows for directly compressing the powdery materials together, forming a solid pharmaceutical composition, into tablets without an intermediate granulating step, allowing to avoid changing the physical (e.g. crystal form) and chemical properties of the drug. The ingredients of the tablet are simply mixed in dry form and compressed in a tablet press.

Roller compaction (RC) is a special way of providing granules for the formulation of solid pharmaceutical compositions (as such or in capsules or after compression in tablets). Avoiding wetting of ingredients offers advantages over wet granulation in processing and, for example, when using moisture sensitive materials. Roller compaction dry granulation process in which the powders containing active ingredients and excipients can agglomerate between the rollers of a compactor. In contrast to wet granulation, roller compaction does not require the use of water or other solvents; therefore, it can be specifically suitable to process compounds that are physically or chemically unstable when exposed to moisture. Solvent granulation with solvents such as ethanol or isopropanol typically requires explosion proof facilities and solvent recycling capabilities, thus, it presents challenges and can be more costly than aqueous granulation. Roller compaction does not require the drying step that is a part of the wet granulation process; therefore, it is advantageous to process compounds that either have a low melting point or degrade rapidly upon heating. On the downside, materials tend to lose bonding strength, or “re-workability,” after being roller compacted. It is commonly found that the tablet hardness of roller compacted materials is much lower than that of the virgin stock under the same tablet compression force. Extremely high roller compaction force not only reduces the “re-workability” but may also cause compact discoloration and/or splitting. In addition, a very high compaction force may reduce the drug dissolution rate, especially for poorly soluble compounds.

Tablets made by roller compaction often show inferior tensile strength compared to tablets prepared by wet granulation or direct compaction. Also, minimum compaction force should normally be used, as well as a smaller particle size of the starting powders.

A second disadvantage of roll compaction known in the art can be the production of non-compacted powder. Especially if no liquid binder is used, high amounts of fines may remain and less product yield is obtained versus wet granulation.

### SUMMARY OF THE INVENTION

Surprisingly, especially in the case of using roller compaction, good densification, flow and scalability aspects were found with TNO155 (especially in its succinate salt form) according to the present invention. Surprisingly, good friability (the tendency of a solid substance to break into smaller pieces under duress or contact, especially by rubbing) was also found. Compositions with high preservation of this active ingredient, under adverse conditions, could be found. The use of the dry tableting methods (roller compaction and direct compression) avoided changes of TNO155 or its salt (in particular the succinate salt as defined below) into different polymorphic forms. This turned out to be an issue when wet granulation was used. However, in some cases the original form might have prevailed or re-establish itself due to the presence of remaining crystals functioning as seed. Direct compression and roller compaction showed comparable disintegration times and friability.

In principle, all granulation and compression techniques examined and their resulting products worked for TNO155 in a more or less acceptable way. However, in spite of the expected disadvantages mentioned above, roller compaction turned out to be most suitable, thus also allowing maintenance of the solid form status without conversions of the crystal form. Further, based on stability studies, it was found that roller compaction offers better densification, flow and scalability aspects over other manufacturing processes.

In summary, feasible pharmaceutical formulations have been established that allow for highly useful pharmacokinetics and pharmacodynamics properties as well as improved storability, manufacture and handling of medicine containing TNO155 as the active ingredient.

TNO155 is known under its chemical name, (3S,4S)-8-(6-amino-5-((2-amino-3-chloropyridin-4-yl)thio)pyrazin-2-yl)-3-methyl-2-oxa-8-azaspiro[4.5]decan-4-amine, and has the formula (which represents the free base form):

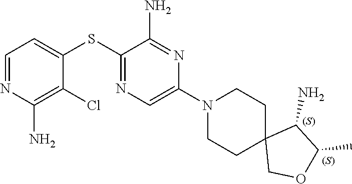

The free base or the pharmaceutically acceptable salts formed from the free base are referred to herein as TNO155 or a pharmaceutically acceptable salt thereof. TNO155 and its manufacture and uses are mentioned in, for example, WO2015/107495 A, see e.g. Example 69.

A preferred pharmaceutically acceptable salt of this compound, (3S,4S)-8-(6-amino-5-((2-amino-3-chloropyridin-4-yl)thio)pyrazin-2-yl)-3-methyl-2-oxa-8-azaspiro[4.5]decan-4-amine, is its 1:1 succinic acid addition salt. The hemihydrate form of this salt can (following the convention of the S/N Guide 2011, European Pharmacopoeia, FIG. A-4-15 under A-4, VI “Amine Salts” with the structure of the amine on the left (as if it were in the amine form) and the structure of the acid (as if it were not dissociated) on the right) be represented by the formula:

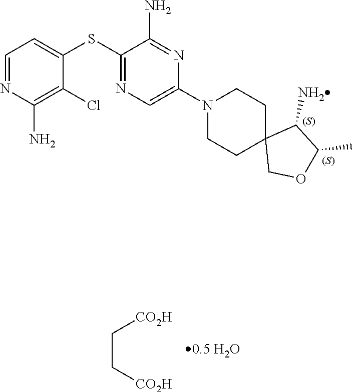

This salt, as well as other pharmaceutically acceptable salts of TNO155, are disclosed in particular in WO2020/065453 A1.

A most preferred variant of this salt is (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) hemihydrate form H_{A}, especially characterized by an X-Ray Powder Diffraction (XRPD) pattern with at least one, two, three or all peaks of the following 2-theta values (±0.2, respectively): 8.1, 16.3, 17.5, 22.5 and 26.8, more preferably one, two, three, four, five, six, seven, eight, nine, ten, eleven or all peaks of the 2-theta values (±0.2, respectively) in the following table:

| Angle (2-theta) | d-value | Rel. intensity |
|---|---|---|
| in deg. | in Å | in % |
| 4.4 | 19.97 | 9.6% |
| 8.1 | 10.85 | 24.9% |
| 16.3 | 5.42 | 57.5% |
| 17.5 | 5.07 | 100.0% |
| 20.9 | 4.24 | 11.1% |
| 22.5 | 3.95 | 41.7% |
| 23.0 | 3.86 | 25.5% |
| 23.7 | 3.76 | 18.3% |
| 24.6 | 3.61 | 23.9% |
| 26.8 | 3.32 | 21.3% |
| 27.9 | 3.20 | 14.8% |
| 36.3 | 2.47 | 15.3% |

or especially an XRPD diagram as shown in FIG. 1 as shown in WO 2020/065453 A1; see especially Example 6 in WO 2020/065453 A1. This specific form is also referred to herein as TNO155 BBA.

All forms (free base TNO155, preferably a pharmaceutically acceptable salt of TNO155 and most preferably TNO155 BBA) fall under the designation “Compound A” which is also used herein.

In a first embodiment, the invention relates to a pharmaceutical formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, and at least one pharmaceutically acceptable excipient, where, in particular, the pharmaceutical formulation is made by a process comprising wet granulation, direct compression or especially roller compaction.

In a second embodiment, the invention relates to a pharmaceutical formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, and at least one pharmaceutically acceptable excipient; said composition obtainable or obtained by a process comprising wet granulation.

In a third embodiment, the invention relates to a pharmaceutical formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, and at least one pharmaceutically acceptable excipient; said composition obtainable or obtained by a process comprising direct compression or roller compaction.

In a fourth embodiment, the invention relates to a pharmaceutical formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, and at least one pharmaceutically acceptable excipient; said composition obtainable or obtained by a process comprising roller compaction.

In a fifth embodiment, the invention relates to a pharmaceutical formulation, especially according to any one of the preceding embodiments, comprising an oral pharmaceutical formulation, especially a tablet, comprising (or especially consisting of) an inner (internal) phase obtainable (especially obtained) from granulation of the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, with one or more pharmaceutically acceptable excipients, where the inner phase is preferably made by a process including wet granulation or especially roller compaction; and an outer (external) phase comprising a mixture of pharmaceutically acceptable excipient; the manufacture including mixing of inner and outer phase and pressing the resulting material to a tablet which is optionally coated. Especially, the granules of the inner phase have a discontinuous distribution (as granules, potentially deformed by pressing the tablet, that is, a grainy distribution) within the outer phase which forms a continuous matrix (except at the outer surface of the tablet where also granule material may be at the outer side that is not completely surrounded by the matrix material). The combination of an inner and an outer phase can be beneficial with regard to (especially tablet) improved disintegration/dissolution, storage robustness and/or tabletability (the capacity of a powdered material to be transformed into a tablet of specified strength under the effect of compaction pressure).

Further embodiments of the invention relate to a pharmaceutical composition according to any other embodiment mentioned herein, where the pharmaceutical composition is a capsule, a sachet or especially a tablet, most especially a coated tablet.

In a specific embodiment, the invention relates to an oral pharmaceutical composition (a composition for oral administration), in particular a tablet, comprising an inner phase with the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form H_{A} as defined in Example 6 of WO2020/065453 A1, and at least one pharmaceutically acceptable excipient, obtainable by roller compaction and at least one pharmaceutically acceptable ingredient, and an outer phase comprising at least one pharmaceutically acceptable ingredient, where in the case of a tablet the outer and inner phase are mixed and pressed to form a tablet, which is uncoated (a core tablet) or coated.

Where mentioned, “obtainable” can be replaced with “obtained”.

Where “less preferred” is used, features after this word are of lower preference than the feature before it.

As pharmaceutical excipients, the following may be mentioned:

A diluent (filler), preferably selected from the group consisting of hydroxyalkylcellulose, especially hydroxypropylmethyl cellulose, e.g. selected from, a sugar alcohol (preferred), such as lactitol, inositol, sorbitol, xylitol or especially mannitol, especially coarser grade mannitol, such as mannitol DC, cellulose, such as microcrystalline cellulose (preferred, e.g. in the outer phase) or cellulose MKGR, (e.g. spray dried) or powdered cellulose, lactose (preferred), e.g. anhydrous lactose or lactose monohydrate, or isomaltose (preferred), starch, hydrolyzed starch, pregelatinized starch, calcium phosphate (e.g. dibasic calcium phosphate or calcium hydrogenphosphate), calcium sulfate, calcium carbonate, magnesium carbonate, kaolin and maltodextrin; or a mixture of two or more such fillers.

In an embodiment, the filler is coarser grade mannitol.

In a further embodiment, the filler is mannitol DC.

A binder, especially selected from a saccharide or disaccharide, such as sucrose or lactose or isomaltose (preferred), copovidone (4-vinylpyrrolidine acetate copolymer) (less preferred), polyvinylpyrrolidone (less preferred), gelatin, cellulose, especially microcrystalline cellulose (most preferred), starch (e.g. paste, mucilage), pregelatinized starch, gelatin, a sugar (e.g. sucrose, glucose, dextrose, molasses, lactose), dextrin, a sugar alcohol, such as xylitol, sorbitol, polymethacrylates, natural and synthetic gums, a cellulose derivative (including a cellulose ether), such as carboxymethyl cellulose, methyl cellulose, hydroxypropyl methyl cellulose (preferred), hydroxypropyl cellulose (preferred), hydroxyethyl cellulose, ethyl cellulose, a wax, magnesium aluminium silicate, and a bentonite; or a mixture of two or more such binders.

In an embodiment, the binder is selected from isomaltose and microcrystalline cellulose.

In a further embodiment, the binder is microcrystalline cellulose.

A disintegrant, especially selected from an effervescent agent, modified cellulose gum, agar, alginic acid, alginate, cross-linked polymer, such as croscarmellose sodium (preferred), crospovidone (polyvinylpyrrolidone; less preferred), (especially low-substituted) hydroxypropyl cellulose (preferred) or sodium starch glycolate).

A glidant, especially selected from a silicium derivative (e.g. colloidal silicon dioxide, colloidal silica, pyrogenic (=fumed) silica, hydrated sodium silico aluminate), talc or magnesium carbonate, especially silicon dioxide (especially fumed silica); or a mixture of two or more such glidants.

In an embodiment, the glidant is fumed silica.

A lubricant (antiadherent), such as talc, a stearate (e.g. magnesium stearate, calcium stearate, zinc stearate, palmitostearate), stearic acid, a hydrogenated vegetable oil, glyceryl behenate, or especially sodium stearyl fumarate, or a mixture of two or more such lubricants.

In an embodiment, the lubricant is stearyl fumarate.

A pharmaceutical formulation according to the invention may comprise further pharmaceutically ingredients, e.g. selected from the group consisting of colorants, absorbents, flavors, sweeteners and desiccants, and/or a coating. A capsule according to the invention can, for example, be a hard gelatin or a soft gelatine capsule. A tablet according to the invention can be without coating or further can carry a coating that is dissolved in the gastrointestinal tract.

Examples for possible coating materials comprise a polymer, a plasticizer and a pigment, such as one or more ingredients selected from polyvinyl alcohol, hydroxypropyl methyl cellulose, talc, polyethylene glycol, lecithin, titanium dioxide, iron oxide yellow and iron oxide red, e.g. enteric release, sustained release or immediate release film coatings; for example, OPADRY®, OPADRY® II, OPADRY® II 85FOPADRY®QX, OPADRY®SGR, OPADRY®ambll, OPADRY®fx, OPADRY®EZ, OPADRY®TF or OPADRY®ENTERIC, such as OPADRY® II white, OPADRY® II yellow, OPADRY® II red or OPADRY® II black, (Colorcon, Ltd, Dartford Kent, England).

It is to be noted that some of the pharmaceutically acceptable excipients can be grouped in more than one of the generic groups (as they can have more than one functional property)—where the generic groups (e.g. diluent, binder, disintegrant, glidant and lubricant) and/or where relative or absolute amounts thereof are mentioned, in case a specific excipient falls under two groups, the minimum and maximum amounts may then be obtained by taking the lowest amount under one generic group up to the added respective maximum amounts in two generic groups. Alternatively, the excipient may be deleted from one of the generic groups in which it is mentioned, leaving only one occurrence.

Where in the following percentages are mentioned in connection with invention embodiments, the amounts refer to the total tablet (including inner and outer phase where given) or the tablet core (including inner and outer phase where given) or single phase tablets where no inner and outer phase are present, totaling up to 100 percentage by weight (wt-%). Coatings add to the weight and may preferably contribute an additional weight, e.g. 1 to 20 wt-%, e.g. 2 to 10 wt-%.

An embodiment of the invention relates to a direct compression tablet, comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form HA as defined in Example 6 of WO2020/065453 A1, preferably in an amount of 5 to 30 wt.-% (=percent by weight wherever mentioned herein, based on the weight of the free base), and at least one pharmaceutically acceptable excipient, especially one or two fillers, a disintegrant, a glidant and a lubricant, preferably one or two fillers especially selected from the group consisting of mannitol (e.g. in an amount of 10 to 60 wt-%, such as 40 to 50 wt-%) and microcrystalline cellulose (e.g. in an amount of 10 to 50 wt-%, such as 25 to 38 wt-%), a disintegrant, especially croscarmellose sodium (e.g. in an amount of 1 to 20 wt-%, such as 3 to 7 wt.-%), a glidant, especially fumed silica (e.g. in an amount of 1 to 15 wt-%, such as 2 to 5 wt-%) and a lubricant, especially magnesium stearate (e.g. in an amount of 0.1 to 3 wt.-%, such as 0.2 to 2 wt-%); which tablet has no coating or has a coating. The percentages refer both to the generic as well as to the specific excipients in this paragraph.

Another embodiment of the invention relates to a tablet comprising an inner phase obtainable by wet granulation, said inner phase including the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form HA as defined in Example 6 of WO2020/065453 A1, preferably in an amount of 5 to 40 wt.-% (=percent by weight wherever mentioned herein, based on the weight of the free base), such as 10 to 30 wt-%, and at least one pharmaceutically acceptable excipient, especially one or two fillers, such as microcrystalline cellulose and/or mannitol, preferably in a total amount of 5 to 60 wt-%, such as 10 to 50 wt-%, a binder, such as hydroxypropyl methylcellulose and/or hydroxypropyl cellulose, especially in an amount from 1 to 15 wt-%, e.g. from 1 to 5 wt-%, a glidant, especially fumed silica, especially in an amount from 1 to 15 wt.-%, e.g. from 1 to 5 wt.-%, and a disintegrant, preferably sodium starch glycolate or croscarmellose sodium, especially in an amount from 1 to 10 wt-%, e.g. 2 to 5 wt-%; and an outer phase which is a mixture comprising a filler, such as microcrystalline cellulose, especially in an amount from 5 to 50 wt.-%, such as 8 to 25 wt.-%, a disintegrant, e.g. croscarmellose sodium or sodium starch glycolate, especially in an amount from 0.5 to 10 wt.-%, e.g. 1 to 3 wt.-%, a glidant, such as fumed silica, especially in an amount from 1 to 10 wt-%, e.g. 1 to 5 wt.-%, and a lubricant, such as magnesium stearate, especially in an amount from 0.1 to 3 wt.-%, such as 0.2 to 2 wt-%. The tablet can be with or without a coating.

Another embodiment of the invention relates to a tablet comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form HA as defined in Example 6 of WO2020/065453 A1, preferably in an amount of 5 to 40 wt.-% (=percent by weight wherever mentioned herein, based on the weight of the free base), one or two fillers, especially selected from mannitol, lactose, calcium hydrogen phosphate and cellulose, especially in an amount of 10 to 60 wt-%, such as 15 to 50 wt.-%, a disintegrant, especially selected from sodium starch glycolate and croscarmellose sodium, especially in an amount of 1 to 10 wt-%, such as 2 to 5 wt-%, a binder, such as hydroxypropyl methyl cellulose, especially in an amount from 1 to 15 wt-%, e.g. from 1 to 5 wt-%, a glidant, such as fumed silica, especially in an amount from 1 to 10 wt-%, e.g. 1 to 5 wt.-%, and a lubricant, such as magnesium stearate, especially in an amount from 0.1 to 3 wt.-%, such as 0.2 to 2 wt-%. The tablet can be with or without a coating.

Another embodiment of the invention relates to a tablet comprising an inner phase obtainable by roller compaction, said inner phase including the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine (also known as TNO155), or a pharmaceutically acceptable salt thereof, especially in the form of a succinate (1:1) salt in hemihydrate form, e.g. form HA as defined in Example 6 of WO2020/065453 A1, preferably in an amount of 5 to 40 wt.-% (=percent by weight wherever mentioned herein, based on the weight of the free base), such as 10 to 30 wt-%, and at least one pharmaceutically acceptable excipient, especially one or two fillers, such as microcrystalline cellulose, and/or mannitol, preferably in a total amount of 5 to 90 wt-%, such as 10 to 80 wt-%, optionally a binder, such as hydroxypropyl methylcellulose and/or hydroxypropyl cellulose, especially in an amount from 0 to 15 wt-%, e.g. 0 wt-% or from 1 to 5 wt-%, a glidant, especially fumed silica, especially in an amount from 1 to 15 wt.-%, e.g. from 1 to 5 wt.-%, a disintegrant, preferably sodium starch glycolate or croscarmellose sodium, especially in an amount from 1 to 10 wt-%, e.g. 2 to 5 wt-%, and a lubricant, such as magnesium stearate, especially in an amount from 0.1 to 3 wt.-%, such as 0.2 to 2 wt-%; and an outer phase which is a mixture comprising a filler, such as microcrystalline cellulose, especially in an amount from 2 to 50 wt.-%, such as 3 to 25 wt.-%, a disintegrant, e.g. croscarmellose sodium or sodium starch glycolate, especially in an amount from 0.5 to 10 wt.-%, e.g. 1 to 4 wt.-%, a glidant, such as fumed silica, especially in an amount from 0.5 to 10 wt-%, e.g. 0.5 to 5 wt.-%, and a lubricant, such as magnesium stearate, especially in an amount from 0.1 to 3 wt.-%, such as 0.2 to 2 wt-%. The tablet can be with or without a coating.

Another invention embodiment relates to a pharmaceutical composition according to any one of the other embodiments, which has a dissolution rate of less than 30 min, especially less than 20 min or preferably 15 min, 10 min 5 min, 4 min, 3 min, 2 min for at least 95% dissolution, measured as described in Example 7.

It was also found that micronized drug substance (TNO155, especially the succinate salt, especially the succinate (1:1) hemihydrate form H_{A}), gave better flowability as a final blend and provided advantage for downstream processing. This is especially true for micronized drug substance having a particle size defined as follows: d10=0.2 μm to 1 μm, especially 0.8 μm; d50=1.0 to 2.0 μm, especially 1.6 μm; and d90=2.1 μm to 5 μm, especially 3.1 μm. For determination of particle size see below.

Another advantageous influence, especially on tablet hardness and weight uniformity, can be established using coarser rather than finer filler, e.g. mannitol. Preferably, the mannitol has a particle size, determined by the Dynamic Image Analysis technique with a Q3 [50%](volume based) in the range from 50 to 250 μm, e.g. from 100 to 220 μm e.g. in accordance with ISO 14488:2007.

The invention also relates to a method of treating an (especially proliferative, especially cancer) disease in an animal, especially human (=patient), in which modulation of SHP2 activity can prevent, inhibit or ameliorate the pathology and/or symptomology of the diseases, which method comprises administering to the animal (especially in need thereof) a pharmaceutical composition as defined herein comprising a therapeutically effective amount of Compound A, alone or in simultaneous or sequential combination with one, two or three additional anti-cancer therapeutics.

The invention also relates to a pharmaceutical composition as defined herein comprising Compound A for use in a method or treating a disease in an animal in which SHP2 activity can prevent, inhibit or ameliorate the pathology and/or symptomology of the disease, said method comprising administering said salt or salt form to a warm-blooded animal, especially a human patient.

The invention also relates to the use of a pharmaceutical composition as defined herein in the manufacture of a medicament for treating a disease in an animal, especially a human patient, in which SHP2 activity contributes to the pathology and/or symptomology of the disease.

As already described in WO 2020/065453 A1, the Src Homolgy-2 phosphatase (SHP2) is a protein tyrosine phosphatase encoded by the PTPN11 gene that contributes to multiple cellular functions including proliferation, differentiation, cell cycle maintenance and migration. SHP2 is involved in signaling through the Ras-mitogen-activated protein kinase, the JAK-STAT or the phosphoinositol 3-kinase-AKT pathways. SHP2 mediates activation of Erk1 and Erk2 (Erk1/2, Erk) MAP kinases by receptor tyrosine kinases such as ErbB1, ErbB2 and c-Met.

SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive conformation, inhibiting its own activity via a binding network involving residues from both the N-SH2 and PTP domains. In response to growth factor stimulation, SHP2 binds to specific tyrosine-phosphorylated sites on docking proteins such as Gab1 and Gab2 via its SH2 domains. This induces a conformational change that results in SHP2 activation.

Mutations in PTPN11 have been identified in several human diseases, such as Noonan Syndrome, Leopard Syndrome, juvenile myelomonocytic leukemias, neuroblastoma, melanoma, acute myeloid leukemia and cancers of the breast, lung and colon. SHP2 is an important downstream signaling molecule for a variety of receptor tyrosine kinases, including the receptors of platelet-derived growth factor (PDGF-R), fibroblast growth factor (FGF-R) and epidermal growth factor (EGF-R). SHP2 is also an important downstream signaling molecule for the activation of the mitogen activated protein (MAP) kinase pathway which can lead to cell transformation, a prerequisite for the development of cancer. Knock-down of SHP2 significantly inhibited cell growth of lung cancer cell lines with SHP2 mutation or EML4/ALK translocations as well as EGFR amplified breast cancers and esophageal cancers. SHP2 is also activated downstream of oncogenes in gastric carcinoma, anaplastic large-cell lymphoma and glioblastoma.

Noonan Syndrome (NS) and Leopard Syndrome (LS)—PTPN11 mutations cause LS (multiple lentigenes, electrocardiographic conduction abnormalities, ocular hypertelorism, pulmonic stenosis, abnormal genitalia, retardation of growth, sensorineural deafness) and NS (congenital anomalies including cardiac defects, craniofacial abnormalities and short stature). Both disorders are part of a family of autosomal dominant syndromes caused by germline mutations in components of the RAS/RAF/MEK/ERK mitogen activating protein kinase pathway, required for normal cell growth and differentiation. Aberrant regulation of this pathway has profound effects, particularly on cardiac development, resulting in various abnormalities, including valvuloseptal defects and/or hypertrophic cardiomyopathy (HCM). Perturbations of the MAPK signaling pathway have been established as central to these disorders and several candidate genes along this pathway have been identified in humans, including mutations in KRAS, NRAS, SOS1, RAF1, BRAF, MEK1, MEK2, SHOC2, and CBL. The gene most commonly mutated in NS and LS is PTPN11. Germline mutations in PTPN11 (SHP2) are found in ˜50% of the cases with NS and nearly all patients with LS that shares certain features with NS. For NS, Y62D and Y63C substitutions in the protein are largely invariant and are among the most common mutations. Both these mutations affect the catalytically inactive conformation of SHP2 without perturbing the binding of the phosphatase to its phosphorylated signaling partners.

Juvenile Myelomonocytic Leukemias (JMML)—Somatic mutations in PTPN11 (SHP2) occur in about 35% of the patients with JMML, a childhood myeloproliferative disorder (MPD). These gain-of-function mutations are typically point mutations in the N-SH2 domain or in the phosphatase domain, which prevent self-inhibition between the catalytic domain and the N-SH2 domain, resulting in SHP2 activity.

Acute Myeloid Leukemia—PTPN11 mutations have been identified in: ˜10% of pediatric acute leukemias, such as myelodysplastic syndrome (MDS); ˜7% of B cell acute lymphoblastic leukemia (B-ALL); and ˜4% of acute myeloid leukemia (AML).

NS and leukemia mutations cause changes in amino acids located at the interface formed by the N-SH2 and PTP domains in the self-inhibited SHP2 conformation, disrupting the inhibitory intramolecular interaction, leading to hyperactivity of the catalytic domain.

SHP2 acts as a positive regulator in receptor tyrosine kinase (RTK) signaling. Cancers containing RTK alterations (EGFRamp, Her2^{amp}, FGFR^{amp}, Met^{amp}, translocated/activated RTK, i.e. ALK, BCR/ABL) include Esophageal, Breast, Lung, Colon, Gastric, Glioma, Head and Neck cancers.

Esophageal cancer (or oesophageal cancer) is a malignancy of the esophagus. There are various subtypes, primarily squamous cell cancer (<50%) and adeno-carcinoma. There is a high rate of RTK expression in esophageal adenocarcinoma and squamous cell cancer. A SHP2 inhibitor of the invention can, therefore, be employed for innovative treatment strategies.

Breast cancer is a major type of cancer and a leading cause of death in women, where patients develop resistance to current drugs. There are four major subtypes of breast cancers including luminal A, luminal B, Her2 like, and triple negative/Basal-like. Triple negative breast cancer (TNBC) is an aggressive breast cancer lacking specific targeted therapy. Epidermal growth factor receptor I (EGFR) has emerged as a promising target in TNBC. Inhibition of Her2 as well as EGFR via SHP2 may be a promising therapy in breast cancer.

Lung Cancer—NSCLC is currently a major cause of cancer-related mortality. accounting for about 85% of lung cancers (predominantly adenocarcinomas and squamous cell carcinomas). Although cytotoxic chemotherapy remains an important part of treatment, targeted therapies based on genetic alterations such as EGFR and ALK in the tumor are more likely to benefit from a targeted therapy.

Colon Cancer—Approximately 30% to 50% of colorectal tumors are known to have a mutated (abnormal) KRAS, and BRAF mutations occur in 10 to 15% of colorectal cancers. For a subset of patients whose colorectal tumors have been demonstrated to over express EGFR, these patients exhibit a favorable clinical response to anti-EGFR therapy.

Gastric Cancer is one of the most prevalent cancer types. Aberrant expression of tyrosine kinases, as reflected by the aberrant tyrosine phosphorylation in gastric cancer cells, is known in the art. Three receptor-tyrosine kinases, c-met (HGFreceptor), FGF receptor 2, and erbB2/neu are frequently amplified in gastric carcinomas. Thus, subversion of different signal pathways may contribute to the progression of different types of gastric cancers.

Neuroblastoma is a pediatric tumor of the developing sympathetic nervous system, accounting for about 8% of childhood cancers. Genomic alterations of the anaplastic lymphoma kinase (ALK) gene have been postulated to contribute to neuroblastoma pathogenesis.

Squamous-cell carcinoma of the head and neck (SCCHN). High levels of EGFR expression are correlated with poor prognosis and resistance to radiation therapy in a variety of cancers, mostly in squamous-cell carcinoma of the head and neck (SCCHN). Blocking of the EGFR signaling results in inhibition of the stimulation of the receptor, cell proliferation, and reduced invasiveness and metastases. The EGFR is, therefore, a prime target for new anticancer therapy in SCCHN.

Malignant peripheral nerve sheath tumors (MPNST) are soft-tissue sarcomas that can occur either sporadically (˜45%), in association with neurofibromatosis type 1 (˜45%), or in association with prior radiotherapy (˜10%). Neurofibromatosis type 1 (NF1) is a common neurogenetic syndrome characterized by neurocognitive effects, a predisposition to develop benign and malignant tumors, cutaneous and other physical findings, and, in 30-50% of patients, plexiform neurofibromas (pNF). pNF are precursor tumors to the malignant counterpart, malignant peripheral nerve sheath tumor (MPNST), and can themselves be a substantial cause of pain, disfigurement and dysfunction. SHP2 inhibition counteracts the RAS-activating effects of NF1 loss. NF1 is involved in de-activating RAS, while SHP2 is involved in activating RAS. SHP2 inhibition (SHP2i) and combination SHP2i can be a strategy to overcome signaling adaptation to, for example, MEKi in tumors with hyperactive RAS due to loss of NF1. SHP2i and combination SHP2i can be a strategy to inhibit inhibitor-induced pathway reactivation to identify optimal therapeutic strategies to effectively target NF1-associated MPNST.

The present invention relates to a pharmaceutical composition comprising Compound A, which composition is capable of inhibiting the activity of SHP2.

In certain embodiments, the present invention relates to the aforementioned method and uses, wherein said SHP2-mediated disorders are cancers selected from, but not limited to: JMML; AML; MDS; B-ALL; neuroblastoma; malignant peripheral nerve sheath tumors (MPNST); esophageal; breast cancer; lung cancer; colon cancer; Gastric cancer, Head and Neck cancer. Other disorders are selected from: NS; LS; JMML; AML; MDS; B-ALL; neuroblastoma; esophageal; breast cancer; lung cancer; colon cancer; gastric cancer; head and neck cancer or any other cancer mentioned above or below.

A pharmaceutical composition of the invention comprising Compound A, may be usefully combined with another pharmacologically active compound, or with two or more other pharmacologically active compounds, particularly in the treatment of cancer. For example, TNO155, or a pharmaceutically acceptable salt thereof, as defined above, may be administered simultaneously, sequentially or separately in combination with one or more (preferably one, two or three) agents selected from antiproliferative agents, e.g. anti-cancer or chemotherapy agents, for example, mitotic inhibitors such as a taxane, a vinca alkaloid, paclitaxel, docetaxel, vincristine, vinblastine, vinorelbine or vinflunine, and other anticancer agents, e.g. cisplatin, 5-fluorouracil or 5-fluoro-2-4(1 H,3H)-pyrimidinedione (5FU), flutamide or gemcitabine. Examples of combination partners are mentioned in WO2015/107495, WO2018/130928, WO2020/065453, WO2020/165732, WO2020/165733, WO2020/165734 and WO2021/171261, which are referred to herein.

Such combinations may offer significant advantages, including synergistic activity, in therapy.

The pharmaceutical composition of the present invention is preferably a solid pharmaceutical composition for oral administration, e.g. a capsule (which may include Compound A and at least one pharmaceutically acceptable excipient as powder, granulate, gel or in the form of minitablets), a tablet, a granulate (e.g. administered by means of a sachet), a powder, or as freeze-dried material.

The pharmaceutical composition preferably comprises or consists of dosage units (e.g. tablets, capsules, sachets) for administration 3 times, 2 times or especially once daily, continuously or with interruption times off. Based on TNO 155 free base content, the amount of Compound A per dosage unit is in the range from 1 to 1000 mg, e.g. from 2 to 250 mg, e.g. from 5 to 200 mg, e.g. from 8 to 150 mg, e.g. from 10 to 80 mg. Higher dosage strengths are also possible with the formulations of the present invention.

The pharmaceutical composition can be used in a method of treatment comprising administering a total dosage of 1 to 1000 mg, e.g. 5 to 400 mg, such as 10 to 320 mg, e.g. distributed in one (QD), e.g. 1 to 320 mg, such as 1.5 to 70 mg), two (BID) (e.g. 10 to 320 mg, such as 30 to 80 mg) or three administrations per day, or twice daily (BID) in a 2 weeks on/1 week off (2 w/1 w) cycle, or QD in a 3 week/1 week cycle (e.g. 30-60 mg) or continuously (e.g. 40 or 50 mg QD); without limiting the possible administrations.

Where particle sizes are given as d10, d50 or d90, this relates to the 10^{th}, 50th or 90^{th} percentile, respectively, meaning the diameter of a sphere at which 10%, 50% or 90%, respectively, of the particles in the sample are smaller. ISO 9276-1:1998(E) (2) specifically indicates that d is interchangeable with x. The particle size, especially of the drug substance TNO155, in particular in form H_{A}, is measured by laser diffraction in a cuvette as wet dispersion, using Fraunhofer diffraction based on volume distribution, using a Sympatec HELOS device.

Any one or more general features in any embodiment or definition mentioned so far may independently of other features or collectively be replaced by one of the more specific definitions of such feature, thus providing further embodiments of the invention.

The following examples serve to illustrate the invention without limiting the scope thereof, while also being specific invention embodiments:

Any definitions of features/abbreviations defined in or for any one of the tables or other passages in the Examples are valid also at other positions where the features/definitions appear, each being defined normally only once.

Any ingredients mentioned specifically e.g. by trademarks or the like may be replaced by equivalent ingredients with the same chemical composition as desired, while the mentioned forms are to be considered as preferred.

### DESCRIPTION OF THE FIGURES

FIG. 1 shows a graph with dissolution data as % drug release over time for 10 mg and 80 mg dosage units (film coated tablets=FCT).

FIG. 2 shows a flow diagram of a manufacturing process for a tablet according to the invention including roller compaction.

FIG. 3 shows a diagram comparing the diameter over volume amounts of two different mannitol grades, Mannitol pH (=Mannitol PH) and Mannitol DC as determined by Dynamic Image Analysis.

FIG. 4 shows the Compression Force Hardness Profiles of TNO155 10 mg FCT of TNO155 BBA formulations.

FIG. 5 shows the Weight Uniformity of about 80 mg FCT comprising 10 mg free base of TNO155 (in the form of TNO155 BBA).

### EXAMPLES

For HPLC in Table 1, the following conditions were applied:
Mobile Phase A: 0.1% Trifluoroacetic acid in Water.
Mobile Phase B: Water:Acetonitrile(50:950 v/v)
Column: Aquity HSS T3,
Column Dimension: Length—100 mm, Internal Diameter—2.1 mm, Particle size—1.8 μm
Flow Rate: 0.5 mL/min
Detection: 220 nm
Column Temperature: 40° C.
Auto sampler Temperature: 22° C.
Injection Volume: 4 μL
Run Time: 13.0 minutes
Program: Gradient

TABLE 1

| Time | Mobile Phase | Mobile Phase |
|---|---|---|
| Min | A(%) | B(%) |
| 0 | 92 | 8 |
| 0.5 | 92 | 8 |
| 6.0 | 70 | 30 |
| 9.0 | 5 | 95 |
| 11.0 | 5 | 95 |
| 11.10 | 92 | 8 |
| 13.0 | 92 | 8 |

For HPLC in Table 2, the following conditions were applied:
Mobile Phase A: 0.05% TFA in Water:MeOH (98:2% V/V)
Mobile Phase B: 0.05% TFA in Water:MeOH(5:95% V/V)
Ghost trap: (DS-HP 2.1 mm ID, 30 mm)
Column: Aquity UPLC HSS T3
Column Dimension: Length—100 mm, Internal Diameter—2.1 mm, Particle size—1.8 μm
Flow Rate: 0.3 mL/min
Auto sampler Temperature: Ambient
Detection: 225 nm
Column Temperature: 30° C.
Injection Volume: 2 μL
Run Time: 30.0 minutes
Gradient:

TABLE 2

| Time | Mobile Phase | Mobile Phase |
|---|---|---|
| (Min) | A(%) | B(%) |
| 0 | 100 | 0 |
| 3.0 | 100 | 0 |
| 21.0 | 0 | 100 |
| 26.0 | 0 | 100 |

### Example 1

### Chemical Compatibility Studies

Binary mixtures were prepared at 1% w/w drug load and were found to be compatible at studied stability conditions except PVP-K30 (Table 3). PVP-K30 exhibited some incompatibility after 4 weeks at 40° C./75% RH open condition. All excipients except PVP-K30 may still be suitable for formulation when used in ratios more realistic in a final formulation (see Example 2).

Drug substance=TNO155 succinate (1:1) hemihydrate, form H_{A}, also named TNO155 BBA hereinafter.

TABLE 3
Results of drug substance alone and binary mixtures
at different storage conditions at 4 weeks.

| | | Assay of | Degradation products¹ | | | | |
|---|---|---|---|---|---|---|---|
| | | active | RRT | RRT | RRT | RRT | |
| Sample | Storage | ingredient | 1.21 | 1.23 | 1.29 | 1.31 | Total |
| Name | conditions | [%] | [%] | [%] | [%] | [%] | [%] |
| Drug | 40° C./75% RH, | 98.6 | — | — | — | — | <0.05 |
| Substance | closed | | | | | | |
| | 40° C./75% RH, | 98.7 | — | — | — | — | <0.05 |
| | open | | | | | | |
| Drug | 40° C./75% RH, | 89.5 | — | — | — | — | <0.05 |
| substance + | closed | | | | | | |
| Mannitol | 40° C./75% RH, | 123.4 | — | 0.33 | — | — | 0.33 |
| | open | | | | | | |
| | 50° C., open | 88.0 | — | — | — | — | <0.05 |
| Drug | 40° C./75% RH, | 98.2 | — | — | — | — | <0.05 |
| substance + | closed | | | | | | |
| Calcium | 40° C./75% RH, | 95.5 | — | 0.17 | — | — | 0.26 |
| dihydrogen | open | | | | | | |
| phosphate | 50° C., open | 92.6 | 0.37 | — | — | — | 0.37 |
| Drug | 40° C./75% RH, | 106.4 | 0.14 | | — | — | 0.21 |
| substance + | closed | | | | | | |
| Avicel | 40° C./75% RH, | | | | | | |
| (microcrystalline | open | 133.0 | 0.21 | 0.30 | — | — | 0.62 |
| cellulose) | 50° C., open | 88.1 | — | — | — | — | <0.05 |
| Drug | 40° C./75% RH, | 104.4 | 0.18 | — | — | — | 0.18 |
| substance + | closed | | | | | | |
| Lactose | 40° C./75% RH, | 66.4 | 0.19 | — | — | — | 0.34² |
| | open | | | | | | |
| | 50° C., open | 86.5 | 0.10 | — | — | — | 0.10 |
| Drug | 40° C./75% RH, | 75.3 | 0.26 | — | — | — | 0.26 |
| substance + | closed | | | | | | |
| Aerosil | 40° C./75% RH, | 94.4 | — | 0.13 | — | — | 0.13 |
| | open | | | | | | |
| | 50° C., open | 76.3 | — | — | — | — | <0.05 |
| Drug | 40° C./75% RH, | 101.8 | — | — | — | — | <0.05 |
| substance + | closed | | | | | | |
| Talc | 40° C./75% RH, | 107.6 | — | — | — | — | <0.05 |
| | open | | | | | | |
| | 50° C., open | 104.0 | — | — | — | — | 0.06 |
| Drug | 40° C./75% RH, | 109.7 | — | — | — | — | 0.09 |
| substance + | closed | | | | | | |
| Magnesium | 40° C./75% RH, | 108.9 | — | — | — | — | 0.17³ |
| stearate | open | | | | | | |
| | 50° C., open | 72.6 | — | — | — | — | <0.05 |
| Drug | 40° C./75% RH, | 124.2 | — | — | — | — | 0.07 |
| substance + | closed | | | | | | |
| Sodium | 40° C./75% RH, | 88.4 | — | — | — | — | 0.06 |
| stearyl | open | | | | | | |
| fumarate | 50° C., open | 86.2 | — | — | — | — | 0.06 |
| Drug | 40° C./75% RH, | 104.3 | 0.07 | — | — | — | 0.07 |
| substance + | closed | | | | | | |
| HPMC 603 | 40° C./75% RH, | 94.1 | 0.26 | — | — | — | 0.62⁴ |
| | open | | | | | | |
| | 50° C., open | 109.3 | — | — | — | — | <0.05 |
| Drug | 40° C./75% RH, | 102.3 | — | — | 0.10 | — | 0.10 |
| substance + | closed | | | | | | |
| PVP K-30 | 40° C./75% RH, | 71.3 | 0.20 | — | 0.91 | 0.11 | 1.49⁵ |
| | open | | | | | | |
| | 50° C., open | 88.9 | — | — | 0.25 | — | 0.25 |

¹Reporting limit for the total degradation is 0.05%.
²Degradation products above 0.10% at RRT 1.14 (0.15%)
³Degradation products above 0.10% at RRT 1.44 (0.11%)
⁴Degradation products above 0.10% at RRT 1.52 (0.20%)
⁵Degradation products above 0.10% at RRT 0.42 (0.11%) and RRT 1.14 (0.11%)
RRT refers to (peak at) relative retention time.

Thus it can be shown that the ingredients mentioned allow for acceptable stabilities, except PVP K-30 which is an example of a less preferable ingredient.

### Example 2

### Compatibility Assays—about 17% Drug Load w/w

Three tablet formulations prepared at more realistic and usual drug load of approx. 17% w/w (Table 4), exhibited acceptable stability in all the conditions (Table 5):

TABLE 4
Compositions of Core table formulations

| | | Wet Granulation | | Wet Granulation | | Direct Compression | |
|---|---|---|---|---|---|---|---|
| | Material | TNO155-ORA-0041 | | TNO155-ORA-0042 | | TNO155-ORA-0040 | |
| Component | No. | mg/Unit | % w/w | mg/Unit | % w/w | mg/Unit | % w/w |
| TNO155 BBA | 832743 | 100.000 | 16.667 | 100.000 | 16.667 | 100.000 | 16.667 |
| Avicel PH101^{a} | 100074 | 129.000 | 21.500 | 108.000 | 18.000 | — | — |
| Cellulose | 100188 | 18.000 | 3.000 | — | — | — | — |
| HPM 603^{b} | | | | | | | |
| Mannitol PH^{c} | 100024 | 200.000 | 33.333 | — | — | — | — |
| Mannitol DC^{d} | 151069 | — | — | — | — | 300.000 | 50.000 |
| Calcium | 100144 | — | — | 221.000 | 36.833 | — | — |
| Hydrogen | | | | | | | |
| Phosphate | | | | | | | |
| HPC-LF^{e} | 970121 | — | — | 18.000 | 3.000 | — | — |
| Aerosil 200 PH^{f} | 100257 | 15.000 | 2.500 | 15.000 | 2.500 | 6.000 | 1.000 |
| Sodium Starch | 100127 | — | — | 18.000 | 3.000 | — | — |
| Glycolate | | | | | | | |
| Croscarmellose | 100141 | 18.000 | 3.000 | — | — | 30.000 | 5.000 |
| sodium | | | | | | | |
| Avicel PH 102^{g} | 103266 | — | — | — | — | 155.000 | 25.833 |
| Magnesium | 100217 | — | — | — | — | 9.000 | 1.500 |
| Stearate | | | | | | | |

External Phase

| | | | | | | | |
|---|---|---|---|---|---|---|---|
| Avicel PH 102 | 103266 | 84.000 | 14.000 | 84.000 | 14.000 | — | — |
| Croscarmellose | 100141 | 12.000 | 2.000 | — | — | — | — |
| sodium | | | | | | | |
| Sodium Starch | 100127 | — | — | 12.000 | 2.000 | — | — |
| Glycolate | | | | | | | |
| Aerosil 200 PH | 100257 | 15.000 | 2.500 | 15.000 | 2.500 | — | — |
| Magnesium | 100217 | 9.000 | 1.500 | 9.000 | 1.500 | — | — |
| Stearate | | | | | | | |
| TOTAL | | 600.000 | 100.000 | 600.000 | 100.000 | 600.000 | 100.000 |

^{a}microcrystalline cellulose (Vivapur 101, JRS pharma), fine size (65 μm average size, laser diffraction, according to manufacturer)
^{b}hydroxypropyl methyl cellulose, Methocel E3 Premium LV Hydroxypropyl Methylcellulose from Dow
^{c}fine mannitol (Pearlitol 160 C, Roquette)
^{d}coarser mannitol compared with ^{c}, Parteck M200 Emprove, Merck was used
^{e}hydroxypropylcellulose 300-600 cps, e.g. Klucel ™ EXF from Ashland
^{f}colloidal silicon dioxide from Evonik Industries
^{g}microcrystalline cellulose (Vivapur 102, JRS Pharma) = Cellulose MK GR, medium size (130 μm average size, laser diffraction, according to manufacturer)
TNO155-ORA-0040, TNO155-ORA-0041 and TNO155-ORA-0042 refer to Batch Numbers of the TNO155 BBA formulation.

Result of table samples of Table 4 at different storage conditions at 4 weeks

TABLE 5
Results of Tablet samples at different storage conditions at 4 weeks

| | | Assay of | Degradation products | | | |
|---|---|---|---|---|---|---|
| | | active | RRT | RRT | RRT | |
| Sample | Storage | ingredient | 1.17 | 1.26 | 1.57 | Total |
| Name | conditions | [%] | [%] | [%] | [%] | [%] |
| TNO155-ORA-0040 | 40° C./75% RH, closed | 102.7 | <0.05 | <0.05 | <0.05 | <0.05 |
| | 40° C./75% RH, open | 99.3 | <0.05 | <0.05 | <0.05 | <0.05 |
| | 50° C., closed | 101.8 | <0.05 | <0.05 | <0.05 | <0.05 |
| | 50° C., open | 98.0 | 0.08 | <0.05 | <0.05 | 0.08 |
| TNO155-ORA-0041 | 40° C./75% RH, closed | 94.9 | 0.07 | <0.05 | <0.05 | 0.07 |
| | 40° C./75% RH, open | 93.2 | 0.15 | 0.08 | <0.05 | 0.23 |
| | 50° C., closed | 91.5 | 0.14 | 0.15 | <0.05 | 0.29 |
| | 50° C., open | 90.6 | 0.27 | 0.27 | 0.14 | 0.68 |
| TNO155-ORA-0042 | 40° C./75% RH, closed | 99.9 | 0.06 | 0.05 | <0.05 | 0.11 |
| | 40° C./75% RH, open | 98.3 | 0.08 | 0.06 | <0.05 | 0.14 |
| | 50° C., closed | 95.0 | 0.11 | 0.19 | <0.05 | 0.30 |
| | 50° C., open | 92.6 | 0.13 | 0.20 | <0.05 | 0.33 |

All three formulations exhibited acceptable stability under the conditions shown.

### Example 3

### Direct Compression Based Compositions

Direct compression (DC) can be used for TNO155 succinate (1:1) hemihydrate form H_{A}, called TNO155 BBA (TNO155-ORA 0040 and TNO155-ORA-0044 refers to different Batch Numbers of formulations hereinafter, see Table 4)

TABLE 6
Compositions for direct compression based TNO155 core tablets

| | | TNO155-ORA- 0040 | | TNO155-ORA- 0044 | |
|---|---|---|---|---|---|
| Composition | Material No. | mg/unit | mg/unit | mg/unit | % w/w |
| TNO155-BBA ¹ | 822743 | 128.000 ² | 21.333 | 128.000 ² | 16.000 |
| Mannitol DC | 151069 | 300.000 | 50.000 | 340.000 | 42.500 |
| Cellulose MK GR | 103266 | 127.000 ³ | 21.167 | 256.000 ³ | 32.000 |
| Crosscarmellose | 100141 | 30.000 | 5.000 | 40.000 | 5.000 |
| sodium | | | | | |
| Aerosil 200 PH | 100257 | 6.000 | 1.000 | 24.000 | 3.000 |
| Magnesium Stearate | 100217 | 9.000 | 1.500 | 12.000 | 1.500 |
| Total | | 600.000 | 100.000 | 800.000 | 100.000 |

¹ S_{A/B} = Salt factor, 1.280 (meaning 128 mg TNO BBA (Salt) is equivalent to 100 mg free base of TNO155,
² Equivalent to 100 mg free base of TNO155,
³ Used for adjusting the quantity of DS compensation.
⁴Cellulose MK GR is microcrystalline cellulose (e.g. Avicel PH 102)

These core tablets, which showed disintegration time (DT) of <4 minutes (measured herein always with 6 tablets, 800 ml water, 37° C.) and friability of <0.40% after 500 revolutions (where friability is measured in the present examples, a 2 drum friabilator, sample weight>6.5 g is used) can be coated into film coated tablets using conventional coating operations.

### Example 4

### Wet Granulation (WG) Based Compositions

Wet granulation can be used for TNO155 BBA film coated tablets (FTC) manufacturing. Core tablets of wet granulation based Batch No. (B.N.) TNO155-ORA-0041 and B.N. TNO155-ORA-0042 (Table 7) showed disintegration time (DT) of <7 minutes and friability of <0.70% after 500 revolutions.

TABLE 7
Composition for WG based TNO155 BBA core tablets:

| | | B.N. TNO155-ORA-0041 | | B.N. TNO155-ORA-0042 | |
|---|---|---|---|---|---|
| Component | Material No. | mg/Unit | % w/w | mg/Unit | % w/w |
| TNO155-BBA ¹ | 832743 | 128.000 ² | 21.333 | 128.000 ² | 21.333 |
| Avicel PH101 | 100074 | 101.000 ³ | 16.833 | 80.000 ³ | 13.333 |
| Cellulose HPM 603 | 100188 | 18.000 | 3.000 | — | |
| Mannitol PH | 100024 | 200.000 | 33.333 | — | |
| Calcium Hydrogen | 100144 | — | | 221.000 | 36.833 |
| Phosphate | | | | | |
| Hydroxypropyl cellulose | 970121 | — | | 18.000 | 3.000 |
| 300-600 cps | | | | | |
| Aerosil 200 PH | 100257 | 15.000 | 2.500 | 15.000 | 2.500 |
| Sodium Starch Glycolate | 100127 | — | | 18.000 | 3.000 |
| Croscarmellose sodium | 100141 | 18.000 | 3.000 | — | |
| Total IG phase | | 480.000 | 80.000 | 480.000 | 80.000 |
| Avicel PH 102 | 103266 | 84.000 | 14.000 | 84.000 | 14.000 |
| Crosscaramellose | 100141 | 12.000 | 2.000 | — | |
| sodium | | | | | |
| Sodium Starch Glycolate | 100127 | — | | 12.000 | 2.000 |
| Aerosil 200 PH | 100257 | 15.000 | 2.500 | 15.000 | 2.500 |
| Magnesium Stearate | 100217 | 9.000 | 1.500 | 9.000 | 1.500 |
| TOTAL | | 600.000 | 100.000 | 600.000 | 100.000 |

¹ S_{A/B}: 1.280,
² Equivalent to 100 mg free base of TNO155,
³ Used for adjusting the quantity of DS compensation

WG based core tablets can easily be converted into film-coated tablets with conventional coating operations. However, a 30% w/w slurry of TNO155-BBA in water showed solid form conversion after 5 hrs. Looking at an unknown landscape of polymorphs for TNO155, WG was not pursued further.

### Example 5

### Roller Compaction (RC) Based Compositions

a) Screening of RC based compositions:

RC was identified as a better manufacturing process because of the specific benefits found, for example, better densification, better flow and better scalability compared with the other manufacturing processes. Four compositions with a drug load of 16.000% (see Table 8) were prepared considering the following aspects:
Material properties (brittle/plastic, aqueous solubility)
Flow aspects
Chemical compatibility of excipients with TNO155 BBA
Drug product compaction descriptors.

TABLE 8
Compositions for RC based TNO155 FCT

| | | B.N. TNO155-ORA- | | B.N. TNO155-ORA- | | B.N. TNO155-ORA- | | B.N. TNO155-ORA- | |
|---|---|---|---|---|---|---|---|---|---|
| | Material | 0047-001 | | 0047-002 | | 0047-003 | | 0047-004 | |
| Composition | No. | mg/unit | % w/w | mg/unit | % w/w | mg/unit | % w/w | mg/unit | % w/w |
| TNO155- | 822743 | 102.400² | 16.000 | 102.400² | 16.000 | 102.400² | 16.000 | 102.400² | 16.000 |
| BBA ¹ | | | | | | | | | |
| Mannitol | 151069 | 224.000 | 35.000 | — | — | — | — | — | — |
| DC | | | | | | | | | |
| Mannitol | 100024 | — | — | 224.000 | 35.000 | — | — | 121.600 | 19.000 |
| PH | | | | | | | | | |
| Lactose | 108862 | — | — | — | — | 224.000 | 35.000 | — | — |
| Gesprueht | | | | | | | | | |
| (=sprayed) | | | | | | | | | |
| Ca-Hyd- | 118225 | — | — | — | — | — | — | 224.000³ | 35.000 |
| Phosphat | | | | | | | | | |
| WSF | | | | | | | | | |
| MCC | 100074 | — | — | 121.600³ | 19.000 | — | — | — | — |
| PH101 | | | | | | | | | |
| Cellulose | 103266 | 121.600³ | 19.000 | — | — | 121.600³ | 19.000 | — | — |
| MK GR | | | | | | | | | |
| Sodium | 100127 | — | — | — | — | 19.200 | 3.000 | 19.200 | 3.000 |
| starch | | | | | | | | | |
| glycolate | | | | | | | | | |
| Croscar- | 100141 | 19.200 | 3.000 | 19.200 | 3.000 | — | — | — | — |
| mellose | | | | | | | | | |
| sodium | | | | | | | | | |
| Cellulose | 100188 | 25.600 | 4.000 | 25.600 | 4.000 | 25.600 | 4.000 | 25.600 | 4.000 |
| HPM ⁵ | | | | | | | | | |
| Aerosil | 100257 | 12.800 | 2.000 | 12.800 | 2.000 | 12.800 | 2.000 | 12.800 | 2.000 |
| 200 PH | | | | | | | | | |
| Magnesium | 100217 | 6.400 | 1.000 | 6.400 | 1.000 | 6.400 | 1.000 | 6.400 | 1.000 |
| Stearate | | | | | | | | | |
| IG Fraction | | 512.000 | 80.000 | 512.000 | 80.000 | 512.000 | 80.000 | 512.000 | 80.000 |
| Total | | | | | | | | | |

¹ S_{A/B}: 1.280,
²Equivalent to 80 mg free base of TNO155,
³Used for adjusting the quantity of DS compensation,
⁴ 20% EG Fraction was not added in the final blends,
⁵ hydroxypropylmethyl cellulose (Methocel E3 Premium LV Hydroxypropyl Methylcellulose from The Dow Chemical Co.)

FCT means Film Coated Tablet.

B.N. TNO155-ORA-0047-001, TN0255-ORA-0047-002, TNO-ORA-0047-003 and TNO155-ORA-0047-004 refer to the respective formulations in Table 8.

These compositions were further compressed at increasing compression forces. Obtained compacts where milled to get granules and tested for flow and dispersibility which was analyzed in accordance with Table 9:

TABLE 9
Decision matrix for selection of compositions for RC based TNO255 FCT development

| | | | | |
|---|---|---|---|---|
| | B.N. TNO155- | B.N. TNO155- | B.N. TNO155- | B.N. TNO155- |
| Parameters | ORA- 0047-001 | ORA- 0047-002 | ORA- 0047-003 | ORA- 0047-004 |

Pre-compression blend

| | | | | |
|---|---|---|---|---|
| Flow ¹ | Poor | Extremely Poor | Extremely Poor | Extremely Poor |

Compacts

| | | | | |
|---|---|---|---|---|
| Compressibility ² | Acceptable | Acceptable | Acceptable | Acceptable |
| | compressibility | compressibility | compressibility | compressibility |
| | was achieved | was achieved | was achieved | was achieved |
| Compactability ³ | Better | Acceptable | Acceptable | Acceptable |
| | compactability | compactability | compactability | compactability |
| | was achieved | was achieved | was achieved | was not |
| | than other | | | achieved |
| | variants | | | |
| Bondability ⁴ | Better | Medium | Medium | Medium |
| | bondability was | bondability was | bondability was | bondability was |
| | achieved than | achieved | achieved | achieved |
| | other variants | | | |
| Manufacturability ⁵ | Better | Acceptable | Acceptable | Acceptable |
| | manufacturability | manufacturability | manufacturability | manufacturability |
| | was achieved than | was achieved | was achieved | was achieved |
| | other variants | | | |
| Observations | No Picking | Picking at lower | Picking at lower | Picking at lower |
| | Noted | punch at all | punch at 5 & 10 | punch at all |
| | | MCF | MCF | MCF |
| Ejection force ⁶ | Acceptable | 500N exceeded | Acceptable | Acceptable |
| | ejection forces | at 55 MPa | ejection forces | ejection forces |
| | till 200 MPa. | | till 200 MPa. | till 200 MPa. |
| Disintegration | Acceptable DT | Acceptable DT | Limit of 15 min | Acceptable DT |
| time (DT) (water, | observed at all | observed at all | exceeded at | observed at all |
| 37° C.) ⁷ | compression | compression | highest 314 MPa | compression |
| | pressures | pressures | | pressures |
| Friability after | 0.64 | 0.76 | 1.28 | 3.36 |
| 500 revolutions | | | | |
| for 15 kN | | | | |
| compacts (%) | | | | |

Granules

| | | | | |
|---|---|---|---|---|
| Flow ¹ | Passable | Passable | Passable | Fair |
| Dispersibility in | Slightly clear | Very turbid | Slightly clear | Very turbid |
| water without | dispersion | dispersion | dispersion | dispersion, big |
| stirring at room | | | | lump at the |
| temperature | | | | bottom |

¹ Target: to achieve “Good” flow with a Carr Index (CI): 11%-15% and Hauser Ratio (HR): 1.12-1.18
² Target: to achieve 15%-25% porosity within a compression pressure range of 150 MPa-250 MPa
³ Target: to achieve 2 MPa tensile strength at compression pressure of 150 MPa
⁴ Target: to achieve 2 MPa tensile strength compacts with min. porosity of 20%
⁵ Target: to achieve linear increase in compact hardness with increasing compression forces
⁶ Target: to achieve ejection forces below 500N till compression pressures of 200 MPa
⁷ Target: Not more than 15 minutes

Dispersibility analysis of granules revealed that granules of B.N. TNO155-ORA-0047-001 and B.N. TNO-ORA-0047-003 produced relatively clearer dispersions than the rest of the compositions. B.N. TNO155-ORA-0047-001 composition emerged as Priority 1 (lead prototype) based on the conducted evaluations covering all the critical parameters mentioned in Table 9.

### Example 7

### Achievement of Flow Improvement

Taking a lead from the B.N. TNO155-ORA-0047-001 composition, various compositions were screened. However, the flow properties of the blend before the RC step were not improving. Rat hole formation is an event in which cohesive powder sticks to walls of the hopper or container and does not move uniformly. Only the materials from the center flows, creating ‘rat hole’ appearance in the powder. This is an important indication of poor flow properties and was evident during the material unloading stage of the RC process during TNO155 development.

In order to eliminate the rat holing and further improve the flow, the below aspects were considered to reach a further optimized composition with Microcrystalline Cellulose PH 200 (Table 10) The percentage weight of the IG part was increased from 80% w/w to 90% w/w Mannitol DC amounts were increased, as it has better flow than Cellulose MKGR (Mannitol DC with Carr's Index: 20.8955% and Hausner Ratio: 1.2642 when compared with Cellulose MK-GR with Carr's Index: 26.2690% and Hausner Ratio: 1.3560).

Microcrystalline Cellulose PH 200 (flowability factor of 9.13) was selected over Cellulose MK-GR (flowability factor of 8.45) to further improve the flow properties of the blend, see Powder Technology 342 (2019) 780-788. Table 8 shows a formulation with MCC PH 200:

TABLE 10
Formulation composition with Microcrystalline Cellulose PH 200

B.N.TNO155-ORA-0052-02

| Material name | % w/w | mg/Unit |
|---|---|---|
| TNO155-BBA.003 ¹ | 16.000 | 102.400 ² |
| Mannitol DC | 45.000 | 288.000 |
| Microcrystalline | 18.000 | 115.200 |
| Cellulose PH200 ³ | | |
| Croscarmellose | 3.000 | 19.200 |
| sodium | | |
| Cellulose HP-M 603 | 4.000 | 25.600 |
| Aerosil 200 PH | 2.000 | 12.800 |
| Magnesium Stearate | 2.000 | 12.800 |
| Total IG part | 90.000 | 576.000 |
| Microcrystalline | 5.000 | 32.000 |
| Cellulose PH200 | | |
| Crosscaramellose | 3.000 | 19.200 |
| sodium | | |
| Aerosil 200 PH | 1.000 | 6.400 |
| Magnesium Stearate | 1.000 | 6.400 |
| Total | 100.000 | 640.000 |

¹ S_{A/B}: 1.280,
² Equivalent to 80 mg free base of TNO155,
³ Used for adjusting the quantity of DS compensation

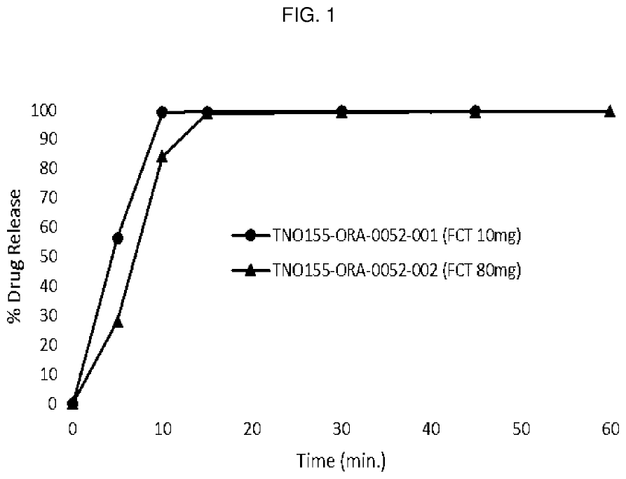

Acceptable compaction descriptors were exhibited by B.N. TNO155-ORA-0052 core tablets of 10 mg (B.N. TNO155-ORA-0052-01, not shown) and 80 mg strength. Adequacy of tensile strength (>2 MPa) was reflected by <1% friability after 500 revolutions (2 drum friabilator, sample wt.: >6.5 g) for both strengths. Porosity values were >5% for both the strengths, which had no impact on DT and dissolution rate (FIG. 1). 10 mg and 80 mg FCTs of B.N. TNO155-ORA-0052 showed >95% release in 15 minutes (0.1 N hydrochloric acid, 500 ml, Basket, 100 rpm).

FIG. 1 shows the dissolution data for the two dosage strengths. Both formulations shown provide good dissolution of more than 95% in less than 15 minutes.

Thus, flow properties were significantly improved with judicious composition changes backed by good scientific rationale. FCT manufacturing was demonstrated along with acceptable drug release profiles.

### Example 8

### Technical Stability of Compositions

Disintegration time (DT) for 10 mg strength and 80 mg strength core tablets was found to be <6 minutes and <8 minutes respectively. While average weights of 10 mg strength and 80 mg strength core tablets was found to be <81 mg and <643 mg respectively. The 6 M satisfactory stability data is captured in Tables 11 to 15.

TABLE 11
Compositions for technical stability study

| | B.N. TNO155-ORA-0056 | | | B.N. TNO155-ORA-0055 | | |
|---|---|---|---|---|---|---|
| | (80 mg Strength) | | | (10 mg Strength) | | |
| | | % w/w | | | % w/w | |
| | | (Core | % w/w | | (Core | % w/w |
| Ingredient | mg/Unit | Tablet) | (FCT) | mg/Unit | Tablet) | (FCT) |
| TNO155.BBA.003 ¹ | 102.400² | 16.000 | 15.550 | 12.800 ³ | 16.000 | 15.148 |
| Mannitol DC | 288.000 | 45.000 | 43.736 | 36.000 | 45.000 | 42.604 |
| Avicel PH200 ⁴ | 121.60 | 19.000 | 18.466 | 15.200 | 19.000 | 17.988 |
| Croscarmellose | 19.200 | 3.000 | 2.916 | 2.400 | 3.000 | 2.840 |
| sodium | | | | | | |
| Cellulose HP-M 603 | 25.600 | 4.000 | 3.888 | 3.200 | 4.000 | 3.787 |
| Aerosil 200 PH | 12.800 | 2.000 | 1.944 | 1.600 | 2.000 | 1.893 |
| Magnesium Stearate | 6.400 | 1.000 | 0.972 | 0.800 | 1.000 | 0.947 |
| IG Total | 576.000 | 90.000 | 87.472 | 72.000 | 90.000 | 85.207 |
| Avicel PH200 | 32.000 | 5.000 | 4.860 | 4.000 | 5.000 | 4.734 |
| Crosscaramellose | 19.200 | 3.000 | 2.916 | 2.400 | 3.000 | 2.840 |
| sodium | | | | | | |
| Aerosil 200 PH | 6.400 | 1.000 | 0.972 | 0.800 | 1.000 | 0.947 |
| Magnesium Stearate | 6.400 | 1.000 | 0.972 | 0.800 | 1.000 | 0.947 |
| Core Tablet Total | 640.000 | 100.000 | 97.191 | 80.000 | 100.000 | 94.675 |
| Opadry II White | 3.330 | | 0.506 | 0.810 | | 0.959 |
| 85F48105.001 | | | | | | |
| Opadry II Yellow | 10.915 | | 1.658 | 2.655 | | 3.142 |
| 85F220148.001 | | | | | | |
| Opadry II Red | 3.330 | | 0.506 | 0.810 | | 0.959 |
| 85F250061.001 | | | | | | |
| Opadry II Black | 0.925 | | 0.140 | 0.225 | | 0.266 |
| 85F277001.001 | | | | | | |
| FCT Total | 658.500 | | 100.000 | 84.500 | | 100.000 |

¹ S_{A/B}: 1.280,
²Equivalent to 80 mg free base of TNO155,
³ Equivalent to 10 mg free base of TNO155,
⁴ Used for adjusting the quantity of DS compensation

TABLE 12
Chemical data by HPLC: 10 mg strength, TNO155-ORA-0055, in
HDPE 175 ml/30's count (30 tablets) with 1 g of desiccant

| | | Assay of | | Degradation products | | | | | | |
|---|---|---|---|---|---|---|---|---|---|---|
| | | active | | RRT | RRT | RRT | RRT | RRT | Max. | |
| | | ingredient | Enantiomer | 1.17 | 1.19 | 1.25 | 1.31 | 1.98 | individual | Total |
| Storage conditions | | [%] | [%] | [%] | [%] | [%] | [%] | [%] | [%] | [%] |
| Requirements | | 90.0-110.0 | ≤0.5 | ≤0.5 | ≤0.5 | ≤0.5 | ≤0.5 | ≤0.5 | ≤0.5 | ≤2.0 |
| Initial analysis | | 96.6 | <0.1¹ | <0.1 | <0.1 | <0.1 | <0.1 | <0.1 | <0.1 | <0.1 |
| 50° C./75% | 1 month | 97.6 | <0.1 | <0.1 | <0.1 | <0.1 | <0.1 | <0.1 | <0.1 | <0.1 |
| RH | | | | | | | | | | |
| 5° C./ambient | 6 months | 99.9 | <0.1 | <0.1 | <0.1 | <0.1 | <0.1 | <0.1 | <0.1 | <0.1 |
| RH | | | | | | | | | | |
| 25° C./60% | 6 months | 98.8 | <0.1 | <0.1 | <0.1 | <0.1 | <0.1 | <0.1 | <0.1 | <0.1 |
| RH | | | | | | | | | | |
| 30° C./75% | 6 months | 98.0 | <0.1 | <0.1 | <0.1 | <0.1 | <0.1 | <0.1 | <0.1 | <0.1 |
| RH | | | | | | | | | | |
| 40° C./75% | 6 months | 97.8 | <0.1 | <0.1 | <0.1 | 0.1 | <0.1 | <0.1 | 0.1 | 0.1 |
| RH | | | | | | | | | | |

¹Reporting threshold: 0.1% for enantiomer and degradation products

TABLE 13
Physical data: 10 mg strength, TNO155-ORA-0055,
HDPE175 ml/30's count with 1 g desiccant

| | | | Disintegration time | | Dissolution after | | |
|---|---|---|---|---|---|---|---|
| | | | [minutes] | | 30 minutes [%] | | Water |
| | | | Average | [min, | Average | [min, | content |
| Storage conditions | | Appearance | (n) | max] | (n) | max] | [%] |
| Initial analysis | | ** | 5.0(6) | [2.7, 6.6] | 96(6) | [94, 98] | 1.8 |
| 50° C./75% | 1 month | No change¹ | N/A | N/A | 99(6) | [97, 102] | 1.8 |
| RH | | | | | | | |
| 5° C./ambient | 6 months | No change¹ | 3.6(4) | [2.8, 4.7] | 96(6) | [95, 99] | 1.3 |
| RH | | | | | | | |
| 25° C./60% | 6 months | No change¹ | 3.3(4) | [2.5, 4.3] | 95(6) | [92, 99] | 1.5 |
| RH | | | | | | | |
| 30° C./75% | 6 months | No change¹ | 3.9(4) | [2.9, 4.8] | 95(6) | [93, 99] | 1.8 |
| RH | | | | | | | |
| 40° C./75% | 6 months | No change¹ | 3.0(3) | [2.5, 3.9] | 96(6) | [94, 98] | 2.2 |
| RH | | | | | | | |

**Brown round tablets with NVR on one side, 8 on other side, debossed, no score
¹No change compared to initial. Brown round tablets with NVR on one side, 8 on other side, debossed, no score

TABLE 14
Chemical data by HPLC: 80 mg, TNO155-ORA-0056,
HDPE 175 ml/30's count with 1 g desiccant

| | | Assay of | | Degradation products | | | | | |
|---|---|---|---|---|---|---|---|---|---|
| | | active | | RRT | RRT | RRT | RRT | Max. | |
| | | ingredient | Enantiomer | 1.19 | 1.25 | 1.31 | 1.98 | individual | Total |
| Storage conditions | | [%] | [%] | [%] | [%] | [%] | [%] | [%] | [%] |
| Requirements | | 90.0-110.0 | ≤0.5 | ≤0.5 | ≤0.5 | ≤0.5 | ≤0.5 | ≤0.5 | ≤2.0 |
| Initial analysis | | 102.1 | <0.11 | <0.1 | <0.1 | <0.1 | <0.1 | <0.1 | <0.1 |
| 50° C./75% | 1 month | 103.8 | <0.1 | <0.1 | <0.1 | <0.1 | <0.1 | <0.1 | <0.1 |
| RH | | | | | | | | | |
| 5° C./ambient | 6 months | 102.0 | <0.1 | <0.1 | <0.1 | <0.1 | <0.1 | <0.1 | <0.1 |
| RH | | | | | | | | | |
| 25° C./60% | 6 months | 102.6 | <0.1 | <0.1 | <0.1 | <0.1 | <0.1 | <0.1 | <0.1 |
| RH | | | | | | | | | |
| 30° C./75% | 6 months | 101.2 | <0.1 | <0.1 | <0.1 | <0.1 | <0.1 | <0.1 | <0.1 |
| RH | | | | | | | | | |
| 40° C./75% | 6 months | 100.4 | <0.1 | 0.1 | 0.1 | <0.1 | <0.1 | 0.1 | 0.2 |
| RH | | | | | | | | | |

¹ Reporting threshold: 0.1% for enantiomer and degradation products

TABLE 15
Physical data: 80 mg, TNO155-ORA-0056, HDPE 175 ml/30's count with 1 g desiccant

| | | | Disintegration time | | Dissolution after | | |
|---|---|---|---|---|---|---|---|
| | | | [minutes] | | 30 minutes [%] | | Water |
| | | | Average | [min, | Average | [min, | content |
| Storage conditions | | Appearance | (n) | max] | (n) | max] | [%] |
| Initial analysis | | ** | 8.8(6) | [8.1, 10.3] | 104(12) | [100, 109] | 1.5 |
| 50° C./75% RH | 1 month | No change¹ | N/A | N/A | 101(6) | [98, 100] | 1.5 |
| 5° C./ambient | 6 months | No change¹ | 6.6(7) | [4.1, 8.4] | 100(6) | [98, 104] | 1.8 |
| RH | | | | | | | |
| 25° C./60% RH | 6 months | No change¹ | 7.7(8) | [6.8, 8.3] | 100(6) | [98, 101] | 1.6 |
| 30° C./75% RH | 6 months | No change¹ | 7.1(8) | [5.5, 8.3] | 101(6) | [97, 106] | 1.7 |
| 40° C./75% RH | 6 months | No change¹ | 8.1(9) | [6.4, 11.7] | 98(6) | [97, 101] | 2.0 |

**Brown ovaloid tablets with NVR on one side, 984 on other side, debossed, no score
¹No change compared to initial. Brown ovaloid tablets with NVR on one side, 984 on other side, debossed, no score

From the data in Table 11 to 15 it follows that all compositions show good stability at the conditions mentioned.

### Example 9

### Manufacturing of a Formulation of TNO155 Film Coated Tablet (FCT)

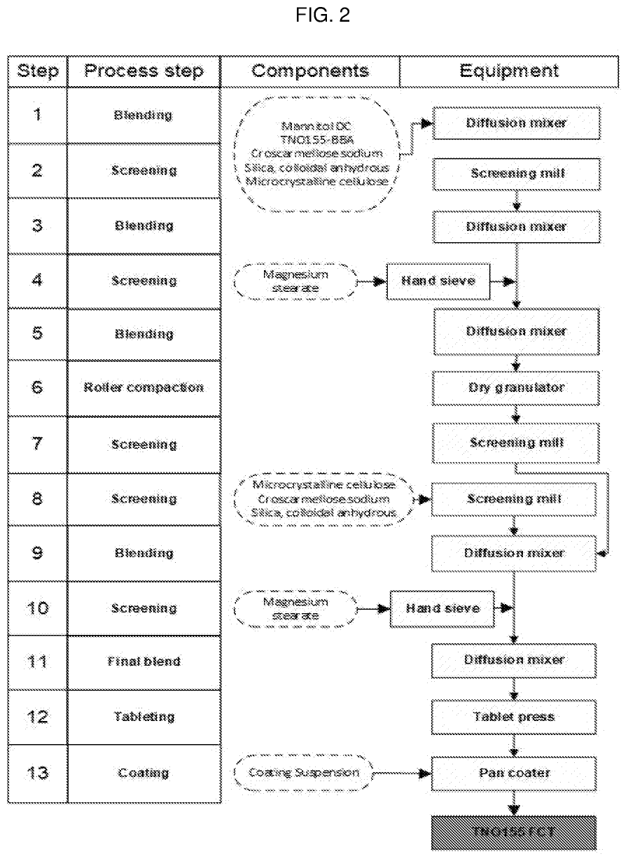

FIG. 2 shows a process flow chart of a process including Roller Compaction for a formulation of TNO155 BBA (TN0155-BBA in the chart).

Two compositions of the following Table 16 (differing in the mannitol used) are manufactured using this process: Table 16 provides the composition evaluated in TNO155-ORA-0073 trials.

TABLE 16
Composition of TNO155-ORA-0073 trials

| | |
|---|---|
| TNO155.BBA/ Excipients | Composition in % |

Inner phase

| | |
|---|---|
| TNO155.BBA | 16.34 |
| Mannitol (PH, | 45.00 |
| or especially DC) | |
| Microcrystalline Cellulose | 24.16 |
| Croscarmellose Sodium | 1.50 |
| colloidal silicon dioxide | 2.00 |
| Magnesium Stearate | 1.00 |
| Outer phase | |
| Microcrystalline Cellulose | 5.00 |
| Croscarmellose Sodium | 3.00 |
| colloidal silicon dioxide | 1.00 |
| Magnesium Stearate | 1.00 |
| Core Tablet | 100 |

The process steps depicted in FIG. 2 are described in the following Table 17: Table 17: Process flow description

| Step | Description |
|---|---|
| 1. Blending | Blending of intra-granular components. Add to the diffusion blender bin |
| | in the following order: MANNITOL, TNO155.BBA, CROSCARMELOSE |
| | SODIUM, COLLOIDAL SILICON DIOXIDE, MICROCRYSTALLINE |
| | CELLULOSE. |
| 2. Screening | Sieving of components from previous step. Use hand sieve or |
| | mechanical mill based on the size of the batch. |
| 3. Blending | Blending of sieved intra-granular components. |
| 4. Sceening | Hand sieving of the intra-granular lubricant: MAGNESIUM STEARATE. |
| 5. Blending | Lubrication; Blending of intra-granular components and sieved lubricant |
| | from previous step. |
| 6. Roller | Roller compaction of lubricated blend from previous step to produce |
| compaction | ribbons with porosity of around 20-35%. |
| 7. Screening | Milling of the ribbons from previous step to produce granules. Pass |
| | using screen. |
| 8. Screening | Addition of the extra-granular components to a bin containing the |
| | granules from the previous step: Pass through a screen |
| | MICROCRYSTALLINE CELLULOSE. CROSCARMELOSE SODIUM |
| | AND COLLOIDAL SILICON DIOXIDE. |
| 9. Blending | Blending of the granules and the extra-granular components. |
| 10. Screening | Hand sieve the extra-granular lubricant into the bin: MAGNESIUM |
| | STEARATE. |
| 11. Blending | Final lubrication; Blend intra and extra-granular components with sieved |
| | lubricant from previous step to produce the final blend. |
| 12. Tableting | Compression of the final blend into cores using tablet press (rotary |
| | press with compression force in the range of 4 to 12 kN). |
| 13. Film coating | Film coating of the cores produced in previous step. |

### Example 10

### Mannitol Grade Selection and its Impact on Manufacturability and Products

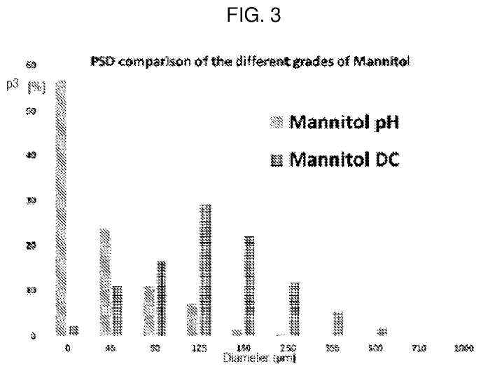

A) Specific grade of Mannitol provides for a better compression hardness profile and suitable flow properties for the tableting process. Two commonly used Mannitol grades were evaluated (which vary with regard to the particle size distribution). FIG. 3 shows the particle size classes obtained.

According to measurement by Dynamic Image Analysis technique (Camsizer, XT Retsch Technology), Mannitol PH (represented as Mannitol pH in FIG. 3) was much finer compared to Mannitol DC grade, it was also found that coarser Mannitol grade (Mannitol DC) provided better flow properties and good hardness profile which was suitable for commercial manufacturing. Measurements of size were carried out using a Camsizer XT (Retsch Technology), for angular particles, 3 g, duration 3 min 33 sec (Mannitol pH)/1 min 41 sec (Mannitol DC), at 0.3% covered area, image rate 1:1, with X-Jet, gap width=4.0 mm, dispersion pressure=10-0 kPa-. For Mannitol pH=PH), Q3 [10.0%]=0.8 μm, Q3 [50.0%]=30.1 μm and Q3 90.0 [%]=119.1 μm. For Mannitol CD, Q3 [10.0%]=79.9 μm, Q3 [50.0%]=162.4 μm and Q3 90.0 [%]=315.8 μm. Q3 [x %] is the percentage (percentile) of the particles at which a given particle diameter is reached, based on volume. In the graph in FIG. 3, the Q3 values are not shown in a cumulative way (cumulative undersize amount) but as partial volumes (p3=fractional volume in a size class) (which add up to Q3).

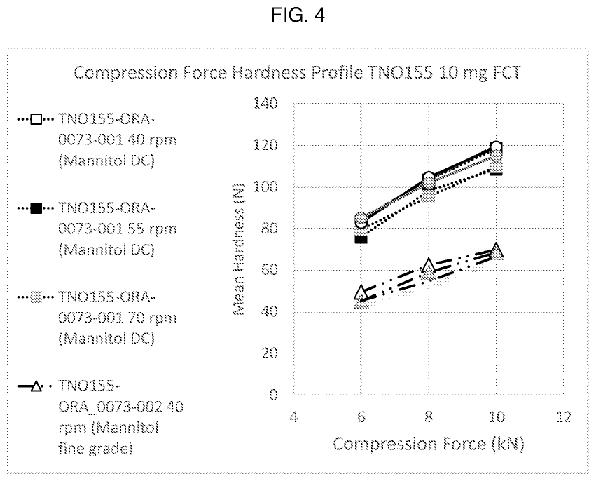

FIG. 4 shows that different Compression Force Hardness Profiles could be found, depending on the mannitol particle size, and exemplified for 10 mg TNO155 BBA compositions. The rpm refer to the rotations per minute of the tabletting device which appears not to have a significant impact. It can be concluded that the coarser materials allow to achieve higher mean hardness at the same compression force. Mannitol fine grade is Mannitol PH.

B) Mannitol grade has influence on the core table weight uniformity:

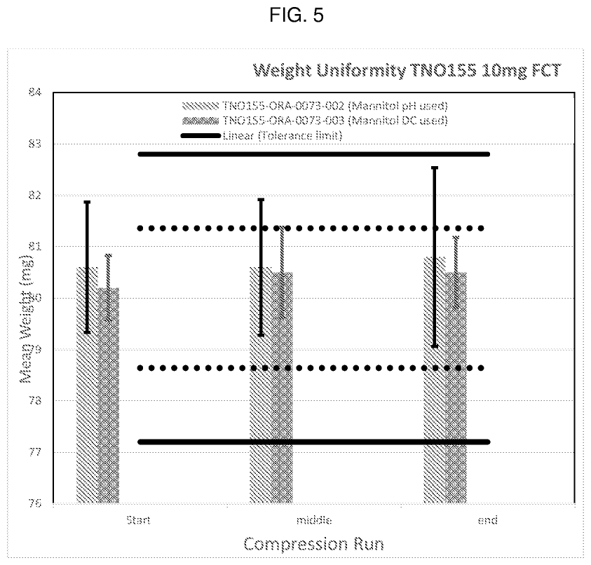

FIG. 5 shows that with coarser Mannitol DC a narrower and thus more defined weight uniformity could be found than with Mannitol PH.

Measurement results are presented as average with error bars for 20 tablets weighing about 80 mg and including 10 mg of TNO155 BBA, respectively.

From A) and B), it can be deduced that a coarser grade of Mannitol (Mannitol DC as an example) offered better manufacturability profile over its finer grade. Even the more stringent control over the weight uniformity at lower strength (worse than higher strength case scenario) demonstrated that Mannitol DC selection would help to ensure the better safety profile for patients.

### Example 11

This Examples provides Film coated Tablet (FCT) composition evaluated for TNO155 10 mg strength, see Table 18.

TABLE 18
10 mg Film-coated tablet

| | |
|---|---|
| | Composition |
| TNO155.BBA/ Excipients | per unit % |

Inner phase

| | |
|---|---|
| TNO155.BBA | 15.148 |
| Mannitol DC | 42.604 |
| Microcrystalline Cellulose ³ | 23.195 |
| Croscarmellose Sodium | 1.420 |
| Colloidal silicon dioxide | 1.893 |
| Magnesium Stearate | 0.947 |
| Inner Phase Total | 85.207 |

Outer phase

| | |
|---|---|
| Microcrystalline Cellulose | 4.734 |
| Croscarmellose Sodium | 2.840 |
| Colloidal silicon dioxide | 0.947 |
| Magnesium Stearate | 0.947 |
| Core Tablet Wt. | 94.675 |
| Opadry Il White | 5.147 |
| Opadry II Yellow | 0.170 |
| Opadry II Red | 0.008 |

1. A pharmaceutical formulation comprising the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and at least one pharmaceutically acceptable excipient, where, in particular, the pharmaceutical formulation is made by a process comprising wet granulation, direct compression or especially roller compaction.

2. The pharmaceutical formulation according to claim 1, wherein the API is (3S,4S)-8-(6-amino-5-((2-amino-3-chloropyridin-4-yl)thio)pyrazin-2-yl)-3-methyl-2-oxa-8-azaspiro[4.5]decan-4-amine in the form of a succinate (1:1) salt in hemihydrate form.

3. The pharmaceutical formulation according to claim 1 or claim 2, obtainable by a process comprising wet granulation.

4. The pharmaceutical formulation according to any one of the preceding claims, obtainable by a process comprising direct compression or roller compaction.

5. The pharmaceutical formulation according to any one of the preceding claims, comprising an inner phase obtained from granulation of the Active Pharmaceutical Ingredient (API) (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form, with one or more pharmaceutically acceptable excipients, where the inner phase is made by a process including wet granulation or roller compaction; and an outer phase comprising a mixture of pharmaceutically acceptable excipients; the manufacture including mixing of inner and outer phase and pressing the resulting material to a tablet which is optionally coated

6. The tablet of claim 5 comprising an inner phase with the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form, and at least one pharmaceutically acceptable excipient, obtainable by roller compaction and at least one pharmaceutically acceptable ingredient, and an outer phase comprising at least one pharmaceutically acceptable ingredient.

7. The pharmaceutical formulation according to any one of the preceding claims, comprising the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form, in an amount of 5 to 30 wt.-%=percent by weight, based on the weight of the free base, and at least one pharmaceutically acceptable excipient, one or two fillers, a disintegrant, a glidant and a lubricant, wherein the one or two fillers are selected from the group consisting of mannitol (e.g. in an amount of 10 to 60 wt-%, such as 40 to 50 wt-%) and microcrystalline cellulose (e.g. in an amount of 10 to 50 wt-%, such as 25 to 38 wt-%), the disintegrant is croscarmellose sodium (e.g. in an amount of 1 to 20 wt-%, such as 3 t0 7 wt.-%), the glidant is fumed silica (e.g. in an amount of 1 to 15 wt-%, such as 2 to 5 wt-%) and the lubricant is magnesium stearate (e.g. in an amount of 0.1 to 3 wt.-%, such as 0.2 to 2 wt-%); which tablet has no coating or has a coating, where the percentages refer to the combination of inner and outer phase without coating.

8. The pharmaceutical formulation according to any one of the preceding claims, which is in the form of a capsule, a sachet or especially a tablet which is uncoated or coated.

9. The pharmaceutical formulation according to any one of claims 1 to 8 in the form of a tablet comprising an inner phase obtainable by wet granulation, said inner phase including the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form in an amount of 5 to 40 wt.-% or 10 to 30 wt-%, and at least one pharmaceutically acceptable excipient, one or two fillers selected from microcrystalline cellulose and mannitol, in a total amount of 5 to 60 wt-%, or 10 to 50 wt-%, a binder selected from hydroxypropyl methylcellulose and hydroxypropyl cellulose, in an amount from 1 to 15 wt-%, or from 1 to 5 wt-%, the glidant fumed silica, in an amount from 1 to 15 wt.-%, or from 1 to 5 wt.-%, and a disintegrant selected from sodium starch glycolate and croscarmellose sodium, in an amount from 1 to 10 wt-%, or 2 to 5 wt-%; and an outer phase which is a mixture comprising the filler microcrystalline cellulose, in an amount from 5 to 50 wt.-%, or 8 to 25 wt.-%, a disintegrant selected from croscarmellose sodium and sodium starch glycolate, in an amount from 0.5 to 10 wt.-%, or 1 to 3 wt.-%, the glidant fumed silica, in an amount from 1 to 10 wt-%, or 1 to 5 wt.-%, and the lubricant magnesium stearate, in an amount from 0.1 to 3 wt.-%, or 0.2 to 2 wt-%; which tablet has no coating or has a coating, where the percentages refer to the combination of inner and outer phase without coating.

10. The pharmaceutical formulation according to any one of claims 1 to 8 in the form of a tablet comprising the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form, in an amount of 5 to 40 wt.-%, one or two fillers, selected from mannitol, lactose, calcium hydrogen phosphate and cellulose, in an amount of 10 to 60 wt-%, or 15 to 50 wt.-%, a disintegrant selected from sodium starch glycolate and croscarmellose sodium, in an amount of 1 to 10 wt-%, or 2 to 5 wt-%, the binder hydroxypropyl methyl cellulose, in an amount from 1 to 15 wt-%, or from 1 to 5 wt-%, the glidant fumed silica, in an amount from 1 to 10 wt-%, or 1 to 5 wt.-%, and the lubricant magnesium stearate, in an amount from 0.1 to 3 wt.-%, or 0.2 to 2 wt-%; which tablet has no coating or has a coating, where the percentages refer to the combination of inner and outer phase without coating.

11. The pharmaceutical formulation according to any one of claims 1 to 8 in the form of a tablet comprising an inner phase obtainable by roller compaction, said inner phase including the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine succinate (1:1) salt in hemihydrate form, in an amount of 5 to 40 wt.-% or 10 to 30 wt-%, and at least one pharmaceutically acceptable excipient, one or two fillers, selected from microcrystalline cellulose, and mannitol, preferably in a total amount of 5 to 90 wt-%, or 10 to 80 wt-%, optionally a binder selected from hydroxypropyl methylcellulose and hydroxypropyl cellulose, in an amount from 0 to 15 wt-%, or from 1 to 5 wt-%, the glidant fumed silica, in an amount from 1 to 15 wt.-%, or from 1 to 5 wt.-%, a disintegrant selected from sodium starch glycolate and croscarmellose sodium, in an amount from 1 to 10 wt-%, or 2 to 5 wt-%, and the lubricant magnesium stearate, in an amount from 0.1 to 3 wt.-%, or 0.2 to 2 wt-%; and an outer phase which is a mixture comprising the filler microcrystalline cellulose, in an amount from 2 to 50 wt.-%, or 3 to 25 wt.-%, a disintegrant selected from croscarmellose sodium and sodium starch glycolate, in an amount from 0.5 to 10 wt.-%, or 1 to 4 wt.-%, the glidant, fumed silica, in an amount from 0.5 to 10 wt-%, or 0.5 to 5 wt.-%, and the lubricant magnesium stearate, in an amount from 0.1 to 3 wt.-%, or 0.2 to 2 wt-%; which tablet has no coating or has a coating, where the percentages refer to the combination of inner and outer phase without coating.

12. The pharmaceutical formulation according to any one of the preceding claims, where the API used for the manufacture of the formulation has a particle size, determined by laser diffraction, defined as follows: d10=0.2 μm to 1 μm or 0.8 μm; d50=1.0 to 2.0 μm or 1.6 μm; and d90=2.1 μm to 5 μm, or 3.1 μm.

13. The pharmaceutical formulation according to any one of the preceding claims, showing a dissolution of 95% in 15 min or less.

14. The pharmaceutical formulation according to any one of claims 1 to 13 for use in the treatment of a proliferative disease.

15. The use of the Active Pharmaceutical Ingredient (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, in the manufacture of a pharmaceutical formulation as defined in any one of claims 1 to 13 for the treatment of a proliferative disease.

16. A method of treating a proliferative, especially cancer disease in a subject, in which modulation of SHP2 activity can prevent, inhibit or ameliorate the pathology and/or symptomology of the diseases, which method comprises administering to the subject in need thereof, a pharmaceutical formulation as defined in any one of claims 1 to 13 comprising a therapeutically effective amount of (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, alone or in simultaneous or sequential combination with one, two or three anti-cancer therapeutics.

17. A method of manufacture of a pharmaceutical composition according to any one of claims 1 to 13, comprising combining (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and at least one pharmaceutically acceptable excipient in a process comprising wet granulation, direct compression or roller compaction.

18. The method according to claim 17 where the pharmaceutical formulation is a tablet, comprising producing an inner granular phase including (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and at least one pharmaceutically acceptable excipient, by roller compaction, and admixing the granules of the inner granular phase with one or more pharmaceutically acceptable excipients to form an outer phase, thus forming a final blend of the inner and outer phase, and compression of the final blend into tablet cores, and leaving the tablets without a coating or coating the tablet cores with a coating material.
